(19) United States
(12) Patent Application Publication
(10) Pub. No.: US 2017/0183672 A1
(43) Pub. Date: Jun. 29, 2017

(54) MAIZE PLASTID TRANSFORMATION METHOD

(71) Applicant: St. John's University, Queens, NY (US)

(72) Inventors: Simon Geir Møller, Kew Gardens, NY (US); Aisling Dunne, Fresh Meadows, NY (US); Nam-Hai Chua, New York, NY (US)

(21) Appl. No.: 15/305,021

(22) PCT Filed: Apr. 17, 2015
(86) PCT No.: PCT/EP2015/058449

§ 371 Date: Oct. 18, 2016

Related U.S. Application Data
(60) Provisional application No. 61/981,386, filed on Apr. 18, 2014.

(51) Int. Cl. C12N 15/82; A01H 4/00

(52) U.S. Cl. CPC C12N 15/821; C12N 15/8207; A01H 4/008; C12N 15/8214; C12N 15/8294

(57) ABSTRACT

The present invention relates to processes for the transformation of plant tissues with a genetic construct which comprises a transgene and a selection gene. The selection gene preferably encodes an auxin biosynthetic polypeptide, thus allowing for selection of transformed plants on media lacking plant auxins. The invention particularly relates to processes wherein the selection step is carried out under a light/dark cycle.

The present invention relates to processes for the transformation of plant tissues with a genetic construct which comprises a transgene and a selection gene. The selection gene preferably encodes an auxin biosynthetic polypeptide, thus allowing for selection of transformed plants on media lacking plant auxins. The invention particularly relates to processes wherein the selection step is carried out under a light/dark cycle.

The use of genetically modified (GM) food crops in agriculture is rapidly increasing with an approximate £14 billion world market in 2005. The production of transgenic plants is, however, a long process which may take a number of weeks. Any steps which speed up this process will therefore be particularly advantageous.

Currently, the standard method used for nuclear transformation of plants such as maize is the method developed by Iowa State University (Frame B et al., In Vitro Cell. Dev. Biol-Plant 36:21-29). In the Iowa State protocol, a plasmid containing a selectable marker and a screenable marker or gene of interest are introduced into immature zygotic embryos or type II callus by particle bombardment. The bombarded tissue (either embryos or callus) is then selected for stable transformation events by transferring the tissue onto selection media which contains the synthetic plant auxin 2,4-D (typically 2 mg/L 2,4-D).

The prior art methods therefore rely on the use of the synthetic plant auxin 2,4-D in the selection media to regenerate the transformed plants and the plants must be kept in the dark for a prolonged period during the initial regeneration phase. This method therefore results in a time-consuming and lengthy process wherein transformed calli emerge after a minimum of 6-8 weeks post-bombardment.

There is therefore a commercial need for methods of regenerating transformed plants, embryos and callus that is quicker and therefore cheaper than the methods currently used.

To circumvent this problem, the invention is based on a selection and regeneration system for plastid transformation based on the use of a hormone-based auxin selection system which allows for the initial selection of putative transformed plant cells, including maize cells, in the dark before transferring to the light.

The Applicant has discovered a new method of transforming plant embryos and callus using selection media that does not contain the synthetic plant auxin 2,4-D and wherein the plant embryos/callus undergo a light/dark cycle during the regeneration phase.

The Applicant's method allows the production of transformed callus at about 3 weeks post-bombardment of embryos or about 4.5 weeks post-bombardment of callus. Transformed embryos and callus can therefore be produced within a much shorter time frame than the currently used methods. The method of the invention is thus quicker for regenerating whole transformed plants, transformed plant embryos and transformed callus.

In one embodiment, the invention provides a process for producing a transformed plant tissue, the process comprising the steps:
(i) transforming plant tissue with a genetic construct,
   wherein the genetic construct comprises a transgene and a selection gene,
   wherein the selection gene encodes an auxin biosynthetic polypeptide; and
(ii) selecting for transformed plant tissue using a light/dark cycle on media which is lacking plant auxin.

Preferably, the selection gene encodes an auxin biosynthetic polypeptide.

In some preferred embodiments, the process comprises:
initiating cell differentiation from a plant tissue; and/or
pre-culturing the plant tissue on osmotic medium prior to the transforming step.

In other preferred embodiments, the process comprises:
a post-transformation recovery interval prior to the selection step.

In yet other preferred embodiments, the process comprises:
regenerating mature somatic embryos to produce shoots/roots,
preferably using a light/dark cycle.

Preferably, the transforming is carried out using a biolistic transformation method.

In a preferred embodiment, the invention provides a process for producing somatic plant embryos, the process comprising the steps:
(i) initiating cell differentiation from immature plant embryos
   (preferably on a callusing medium, preferably comprising auxin);
(ii) pre-culturing the immature plant embryos
   (preferably on an osmotic medium)
   (preferably in the dark);
(iii) transforming the immature plant embryos with a genetic construct
   (preferably using a biolistic transformation method),
   wherein the genetic construct comprises a transgene
   and a gene encoding one or more auxin biosynthetic polypeptides;
(iv) optionally culturing the immature plant embryos
   (preferably on a callusing medium);
(v) selecting for transformed immature plant embryos on media which is lacking plant auxin
   (preferably on a medium lacking 2,4-D)
   (preferably in the dark); and
(vi) selecting mature somatic embryos on media which is lacking plant auxin optionally using a continuous light cycle and then
   using a light/dark cycle,
   wherein the optional continuous light cycle is preferably for about 3 days
   and wherein the light/dark cycle is preferably approx. 16 hour light/8 hour dark cycle for 2-8 days, more preferably for about 6 days.

Preferably, the plant is maize.

In a further preferred embodiment, the invention provides a process for producing a transformed plant, the process comprising the steps:
(i) initiating cell differentiation from immature plant embryos to produce plant calli
   (preferably on a callusing medium, preferably comprising auxin);
(ii) pre-culturing the plant calli
   (preferably on an osmotic medium)
   (preferably in the dark);
(iii) transforming the plant calli with a genetic construct
   (preferably using a biolistic transformation method),
   wherein the genetic construct comprises a transgene
   and a gene encoding one or more auxin biosynthetic polypeptides;
(iv) optionally culturing the bombarded plant calli
   (preferably on an osmotic medium);
(v) selecting for transformed plant calli on media which is lacking plant auxin
   (preferably on a medium lacking 2,4-D)
   (preferably in the dark); and
(vi) selecting plant calli on media which is lacking plant auxin
   using a light/dark cycle,
   wherein the light/dark cycle is preferably approx. 16 hour light/8 hour dark cycle, preferably for 2-8 days, more preferably for about 6 days.
      and preferably regenerating a transformed plant from the calli.

Preferably, the plant is maize.

The method of the invention is suitable for all plants that can be transformed and regenerated, and for which auxin is essential for plant regeneration.

The plant may be a monocot or dicot.

Examples of suitable plants are cereals (rice, wheat, barley, oats, sorghum, corn), legumes (alfalfa, lentils, peanut, pea, soybean), oil crops (palm, sunflower, coconut, canola, olive), cash crops (cotton, sugar cane, cassava), vegetable crops (potato, tomato, carrot, sweet potato, sugar-beet, squash, cucumber, lettuce, broccoli, cauliflower, snap bean, cabbage, celery, onion, garlic), fruits/trees and nuts (banana, grape cantaloupe, muskmelon, watermelon, strawberry, orange, apple, mango, avocado, peach, grapefruit, pineapple, maple, almond), beverages (coffee, tea, cocoa), and timber trees (oak, black walnut, sycamore). Other suitable plants include mosses and duckweed. Preferably, the plant is tobacco or lettuce.

In some embodiments, the plant is rice, soybean, canola, cotton, potato, tomato, carrot, lettuce, cauliflower, cabbage and tobacco.

In other embodiments, the plant is carrot, rice lettuce, cabbage, potato, tomato, oilseed rape, maize, wheat, oats, rye, sugar beet, cotton, sorghum or sugarcane.

Preferably, the plant is maize.

Plant embryos are parts of seeds which contain precursor tissues that eventually develop into leaves, stems and roots, as well as one or more cotyledons.

The plant tissues which are being transformed may be used in any convenient form, for example, as individual cells, groups of cells, in dissociated form or undissociated form, or as part of a plant part. Preferably, the tissues are present in leaves that are removed from intact plants. It is preferable to use actively-growing leaves.

In some embodiments, the plant tissue is a plant embryo or plant callus.

In a preferred embodiment, the genetic construct is targeted to plastids within the plant tissue.

For example, homologous recombination elements may be used which are capable of directing the integration of the genetic construct, or a part thereof, into the genome of at least one plastid which is present in the plant tissue. The homologous recombination elements may, for example, flank the transgene and/or selection gene.

The term “plastid” is intended to cover all organelles which are found in the cytoplasm of eukaryotic plants, which contain DNA, which are bounded by a double membrane, and develop from a common type, i.e. a proplastid. Plastids may contain pigments and/or storage materials.

Examples of plastids include chloroplasts, leucoplasts, amyloplasts, etioplasts, chromoplasts, elaioplasts and gerontoplasts. Preferably, the plastid is a green plastid, most preferably a chloroplast.

The genetic construct comprises a transgene and a selection gene.

As used herein, the term “genetic construct” refers to a nucleic acid molecule comprising the specified elements. The genetic construct may, for example, be in the form of a vector or a plasmid. It may also contain other elements which enable its handling and reproduction, such as an origin of replication, additional selection elements, and multiple cloning sites. Generally, the genetic construct will be a double-stranded nucleic acid molecule, preferably a dsDNA molecule.

In the context of the present invention, the term “transgene” is used to refer to a nucleic acid molecule which is being introduced into the genome of the plant. The transgene may, for example, be a genomic DNA, cDNA or synthetic nucleic acid molecule coding for a peptide or polypeptide; a nucleic acid molecule encoding a mRNA, tRNA or ribozyme; or any other nucleic acid molecule.

Examples of transgenes include those coding for antibodies, antibiotics, herbicides, vaccine antigens, enzymes, enzyme inhibitors and design peptides.

Single or multiple antigens may be produced from viridae, bacteria, fungi or other pathogens. The antigens may be expressed as single units or as multiple units of several antigens, e.g. for broad-spectrum vaccine development.

Enzymes may be produced for use in cosmetics (e.g. superoxide dismutase, peroxidase, etc.). Enzymes may also be produced for use in detergent compositions.

The invention particularly relates to the production of proteins/enzymes with specific activities, for example, immunostimulants to boost immune responses, such as interferons; and growth factors, e.g. transforming growth factor-beta (TGF-beta), bone morphogenic protein (BMP), neurotrophins (NGF, BDNF, NT3), fibroblast growth factor (FGF), proteolytic enzymes (papain, bromelain), and food supplement enzymes (protease, lipase, amylase, cellulase).

The invention also relates to the production or overexpression of proteins/enzymes in plant tissues that make the plants more resistant to biotic and abiotic stress, such as salts and metals. Examples of this include the generation of transplastomic plants that chelate iron (Fe) for mopping up excess metal in agriculturally important areas for future planting.

The invention further relates to the use of transgenes encoding polypeptides which modify fatty acid biosynthesis in plastids.

One or more transgenes may be inserted in the genetic construct. Preferably, the transgene sequences are contiguous.

The transgene sequence may additionally encode a protein purification tag fused to the polypeptide of interest. Examples of protein purification tags include the N-terminal influenza haemagglutinin-HA-epitope (HA) and a sequence of six histidine amino acids (HIS6) and the Strep tag. Each of the transgene products may have a different affinity tag.

The selection gene is preferably one which encodes one or more plant auxin biosynthetic polypeptides. The expression of this transgene results in the production of auxin within the plant.

The auxin biosynthetic polypeptides may be any polypeptides which are involved in the synthesis of a plant auxin or other plant growth regulator, or which regulate the production or metabolism of a plant auxin or other plant growth regulator.

Preferably, there are nucleotide sequences encoding 1, 2, 3, or 4 auxin biosynthetic polypeptides. The nucleotide sequences encoding the auxin biosynthetic polypeptides may be present in an operon, with a single optional promoter and terminator element. Alternatively, the auxin biosynthetic polypeptides nucleotide sequences may each have their own promoters and terminator elements. A further option is that two or more of the nucleotide sequences encoding the auxin biosynthetic polypeptides are present as fusion proteins, optionally with a short linker sequence joining the proteins (e.g. encoding a 1-10 amino acid linker sequence, e.g. a poly-glycine linker). In other embodiments, some of the nucleotide sequences encoding the auxin biosynthetic polypeptides may be present in an operon and/or as fusion proteins, and others have their own promoters and/or terminators.

The nucleotide sequences encoding the auxin biosynthetic polypeptides may be from any suitable source. Due to codon usage, bacterial genes are preferred, because nuclear genes may not be expressed to maximum levels in chloroplasts.

Preferably, the nucleotide sequence encoding the auxin biosynthetic polypeptides is from Agrobacterium tumefaciens or from a plant (e.g. from the plant which is being transformed).

In some preferred embodiments, the or a auxin biosynthetic polypeptides is iaaH (indoleacetamide hydrolase) and/or iaaM (tryptophan mono-oxygenase), which are enzymes involved in auxin biosynthesis. The nucleotide sequences may be from any source. Due to codon usage, bacterial iaaH and/or iaaM genes are preferred. Preferably, the iaaH and/or iaaM nucleotide sequences are from Agrobacterium tumefaciens.

In other embodiments, the auxin biosynthetic polypeptides are selected for the group consisting of AMI1, TAA1, TAR1, TIR2, YUC, AAO1, CYP79B2 and TDC.

The transgene and/or a selection gene may be flanked by homologous recombination elements that are capable of directing the integration of the transgene and/or a selection gene into the genome of the plant tissue.

Upon transformation of the genetic construct into the plant cells the plant tissue, the first and second homologous recombination elements recombine with corresponding sequences in the genome of the selected cells, resulting in the insertion of the transgene and/or a selection gene into the genome of the cells.

The homologous recombination elements may target the transgene and/or a selection gene to the plant nuclear genome, mitochondrial genome or plastid genome, preferably to the plastid genome.

The nucleotide sequences of the homologous recombination elements are selected such that the transgene and/or a selection gene is specifically targeted to one or more selected genomes. In particular, the nucleotide sequences of the homologous recombination elements may be selected such that no or essentially no transgenes and/or a selection genes become integrated into the nuclear genome of the plant or into the mitochondrial genome of the plant. In other words, the nucleotide sequences of the homologous recombination elements may be preferably plastid-specific, i.e. corresponding sequences might not present in the nuclear genome and preferably not present in the mitochondrial genome of the plant in question. This may be done by avoiding sequences which are present in the nuclear genome of the plant and optionally in the mitochondrial genome. The skilled person will readily be able to detect whether a specific sequence is or is not present in the nuclear genome by standard means, for example, by Southern Blotting of the nuclear genome with a labelled sequence probe or by sequence analysis.

Apart from the above, any sequences can be used from the genome as long as the selected insertion site is not lethal to the cell, i.e. it does not result in the death of the cell. Preferably, the insertion sites are not in coding regions of genes.

The orientation of the sequences of the first and second homologous recombination elements should be the same as the orientation in the plant cell genome to allow for efficient homologous recombination.

In order to target the transgene and/or a selection gene to the plastid genome, the nucleotide sequences of the first and second homologous recombination elements must be identical or substantially identical to sequences in the genome of the selected plant plastid.

In the context of the present invention, the term “substantially identical” means that the nucleotide sequences of the first and second homologous recombination sequences are independently more than 95%, preferably more than 98% or more than 99% and particularly preferably 100% identical to sequences which are present in the genome to be transformed. Percentage sequence identities may be determined using the Clustal method of alignment with default parameters, e.g. KTUPLE 1, GAP PENALTY=3, WINDOW=5 and DIAGONALS SAVED=5.

Similarly, the nucleotide sequences of the first and second homologous recombination elements should preferably not be identical or substantially identical to sequences in the nuclear genome of the selected plant, if targeting to the nuclear genome is to be avoided. In this context, the term “substantially identical” means that the nucleotide sequences of the first and second homologous recombination sequences are independently less than 50%, more preferably less than 70% or less than 90% identical to sequences which are present in the nuclear genome of the plant to be transformed. Percentage sequence identities may be determined using the Clustal method of alignment with default parameters, e.g. KTUPLE 1, GAP PENALTY=3, WINDOW=5 and DIAGONALS SAVED=5.

Preferably, the lengths of first and second homologous recombination sequences will independently be 50-2500, 50-2000, 50-1500 or 50-1000 nucleotides each, more preferably about 150, about 1000 or about 1200 nucleotides in length.

The distance between the first and second homologous recombination sequences in the plant genome may be 0-4000 nucleotides or more. Preferably, the distance is about 1-100, 100-500, 500-1000 or 1000-3000 nucleotides.

The total length of the genetic elements which are present between the first and second homologous recombination is preferably less than 4000 nucleotides.

Preferably, the first homologous recombination sequence is nucleotides 68231-69454 of the Zea mays (accession no. NC_001666.2) chloroplast genome DNA; and/or preferably, the second homologous recombination sequence is nucleotides 69455-71184 of the Zea mays (accession no. NC_001666.2) chloroplast genome DNA.

In yet other embodiments, the first homologous recombination sequence is preferably nucleotides 123821-124699 of the Zea mays (accession no. NC_001666.2) choloroplast genome DNA; and/or the second homologous recombination sequence is nucleotides 124764-125784 of the Zea mays (accession no. NC_001666.2) choloroplast genome DNA.

Other preferred pairs of first and second homologous recombination sequences in Zea mays accession no. NC_001666.2 include the following:

(a) 94859-94930 and 95161-96651;
(b) 131265-133501 and 130495-130965;
(c) 13073-13146 and 12509-12579;
(d) 127807-127878 and 126086-127576;
(e) 89236-91472 and 91772-92242;
and
(f) 98038-98916 and 96953-97973.

Prior to the transformation step, the process optionally comprises:
initiating cell differentiation of the plant tissue, and/or
pre-culturing the plant tissue in osmotic medium.

The plant tissue (e.g. immature embryos) may be placed on callusing medium.

Callusing medium can be used to initiate plant cell differentiation. This helps to facilitate the transformation step.

The Callusing Medium may contain an auxin (e.g. 2,4-D).

One example of Callusing Medium is N6E (see Appendix 1).

The step of initiating cell differentiation of the plant tissue is preferably carried out in the dark.

The step of initiating cell differentiation of the plant tissue is preferably carried out for 2 days to 8 weeks.

In embodiments of the invention wherein the plant tissue is immature embryos, the step of initiating cell differentiation of the plant tissue is preferably carried out for 1-4 days, preferably 2-3 days.

In embodiments of the invention wherein the plant tissue is callus, the step of initiating cell differentiation of the plant tissue is preferably carried out for 5-9 weeks, preferably 6-8 weeks.

The step of initiating cell differentiation of the plant tissue is preferably carried out at 21-32° C., preferably at about 28° C.

The plant tissue may also be pre-cultured in Osmotic Medium prior to the transformation step. Osmotic medium is used to reduce turgor pressure in the plant cells.

An osmotic agent may be used (e.g. sorbitol and/or mannitol) to increase gene expression by reducing turgor pressure in cells. This increases the chance of cell survival by avoiding leakage following the shock wave created during bombardment (Rosillo, G., J. Acuña, A. Gaitan & M. Peña De. (2003). “Optimized DNA delivery into Coffea arabica suspension culture cells by particle bombardment”. Plant Cell Tiss. Org. Cult. 74: 45-49). In addition, it is thought that a high concentration of osmotic agents may also induce changes in cell membranes, leading to increased cell tolerance to biolistic delivery impact (Ingram, H. M., J. B. Power, K. C. Lowe & M. R. Davey. (1999). “Optimization of procedures for microprojectile bombardment of microspore-derived embryo in wheat”. Plant Cell Tiss. Org. Cult. 57: 207-210).

One example of Osmotic Medium is N6OSM (see Appendix 1).

The pre-cultured in Osmotic Medium step is preferably carried out in the dark or under reduced light conditions.

For plant embryos, the pre-cultured in Osmotic Medium step is preferably carried out for 2-6 hours, preferably about 4 hours.

For plant calli, the pre-cultured in Osmotic Medium step is preferably carried out for 4-26 hours, preferably about 24 hours.

The person skilled in the art will be aware of numerous methods for transforming plant cells with nucleic acid vectors. These include direct DNA uptake into protoplasts, PEG-mediated uptake to protoplasts, microparticle bombardment, electroporation, heat-shock, micro-injection of DNA, micro-particle bombardment of tissue explants or cells, vacuum-infiltration of plant tissues, and T-DNA mediated transformation of plant tissues by Agrobacterium, and plant (preferably maize) liquid cultures.

The transformation method may target the plant nucleus or plastids.

Preferably, the plastids within the plant tissue are transformed. Any such suitable method may be used.

For targeting the genetic construct to plastids, biolistic transformation is preferred. This involves shooting nucleic acid vector-coated gold particles (micro-projectiles) into plastids of plant tissues, followed by selection of the transformed plastids and plant regeneration. Preferably, the plant tissue is immature embryos or callus.

In some embodiments of the invention, the plant cells to be transformed are guard cells, i.e. stomatal guard cells. Such cells have been shown to be totipotent and therefore regeneration should be more efficient. Guard cells may be used as epidermal strips or as isolated guard cell protoplasts. (Hall et al. 1996. 112 889-892, Plant Physiology; Hall et al. 1996, 14. 1133-1138, Nature Biotechnology).

In some embodiments of the invention, the transformation step is followed by a recovery interval.

Preferably, the recovery interval is 12-60 hours, more preferably, 24-48 hours.

If immature embryos are being transformed, the recovery interval is preferably about 48 hours.

If callus is being transformed, the recovery interval is preferably about 24 hours.

The recovery step is preferably carried out in the dark or under low-light conditions.

Preferably, the plant tissue is maintained on Osmotic Medium after the transformation step.

In some embodiments of the invention, the plant tissue is placed on Callusing Medium prior to the selection step for 4-10 days, preferably for about 7 days.

Preferably, this step is carried out in the dark.

Embodiments of the invention which involve biolistic transformation, the particle bombardment uses helium under high pressure to deliver DNA coated gold micro-particles to target cells. This results in damage to target tissue inflicted by the high-pressure helium. The recovery period (continuing callus formation) post bombardment is thought to allow plant tissue time to recover from this damage and may result in a higher transformation efficiency.

In the selection step, transformed plant tissue is selected for on media which is lacking plant auxin using a light/dark cycle. This is significantly different from standard transformation protocols which require an auxin (e.g. 2,4-D) to initiate shoot development.

In the process of the invention, transformed plants express an auxin biosynthetic polypeptide. Hence the transformed plants of the invention do not need to be selected for on a medium which contains an auxin.

As used herein, the term “lacking auxin” is intended to mean that the selection medium does not contain sufficient auxin to enable the production of shoots and/or the regeneration of the plant. Hence the selection media may still contain trace amounts of auxin.

In some embodiments of the invention, the selection step is carried out in the absence of antibiotics.

In other embodiments of the invention, the selection step is carried out in the absence of spectinomycin.

In other embodiments of the invention, the selection step is carried out in the absence of bialaphos.

In particular, the selection medium is lacking any of the following:
2-4-dichlorophenoxyacetic acid (2,4-D)
3-indoleacetic acid (IAA)
4-chloro indoleacetic acid
indole-3-butyric acid (IBA)
1-naphthaleneacetic acid (NAA)
2,4,5-Trichlorophenoxyacetic acid (2,4,5-T)
Phenylacetic acid (PAA)
4-chloroindole-3-acetic acid (4-CI-IAA),
2-methoxy-3,6-dichlorobenzoic acid (dicamba)
4-amino-3,5,6-trichloropicolinic acid (tordon or picloram).
2,4,5-T, 2-methyl-4-chlorophenoxyacetic acid (MCPA),
2-(2-methyl-4-chlorophenoxy)propionic acids (mecoprop, MCPP),
2-(2,4-dichloropheoxy)propionic acid (dichloroprop, 2,4-DP)
(2,4-dichlorophenoxy)burytic acid (2,4-DB).

In embodiments of the invention where the plant tissue are immature embryos, the plant tissue is preferably transferred to selection medium without auxin about 6-8 days, preferably about 7 days, after transformation.

Preferably, the immature embryos undergo a three-stage selection process:

Preferably, the first selection step is carried out in the dark or under reduced light conditions.

Preferably, the first selection step is carried out at 21-32° C., more preferably at about 28° C.

Preferably, the first selection step is carried out for 6-8, more preferably about 7 days.

Preferably, a second selection step takes place straight after the first selection step or within 1-2 days of the first selection step.

In the second selection step, the plant tissues are placed under continuous light for 2-4 days, preferably for about 3 days.

Preferably, a third selection step takes place straight after the second selection step or within 1-2 days of the second selection step.

In the third selection step, the plant tissues are placed under a light/dark cycle.

Preferably, out of a 24 hour cycle, the light is on for 14-18 hours, more preferably on for about 16 hours.

Preferably, out of a 24 hour cycle, the dark is for 6-10 hours, more preferably for about 8 hours.

Preferably, the third selection step is carried out for 4-8 days, preferably about 6 days.

In the case of transformed embryos, the light/dark cycle selection step is preferably completed less than 4 weeks, more preferably less than 3 weeks and most preferably less than 2 weeks after transformation.

In the case of transformed embryos, green calli are preferably produced less than 4 weeks, more preferably less than 3 weeks and most preferably less than 2 weeks after transformation.

In embodiments of the invention where the plant tissue are calli, the plant tissue is preferably transferred to selection medium without auxin about 12-26 hours, preferably about 24 hours, after transformation.

Preferably, the calli undergo a two-stage selection process:

Preferably, the first selection step is carried out in the dark or under reduced light conditions.

Preferably, the first selection step is carried out at 21-32° C., more preferably at about 28° C.

Preferably, the first selection step is carried out for 6-8 days, more preferably about 7 days.

The first selection step may also be carried out for 3-5 weeks, preferably for about 4 weeks.

Preferably, a second selection step takes place after the first selection step.

In the second selection step, the plant calli are placed under a light/dark cycle. Preferably, out of a 24 hour cycle, the light is on for 14-18 hours, more preferably on for about 16 hours.

Preferably, out of a 24 hour cycle, the dark is for 6-10 hours, more preferably for about 8 hours.

Preferably, the second selection step is carried out for 4-8 days, preferably about 6 days.

In the case of transformed calli, the light/dark cycle selection step is preferably completed less than 6 weeks, more preferably less than 5 weeks and most preferably less than 4 weeks after transformation.

In the case of transformed calli, green calli are preferably produced less than 6 weeks, more preferably less than 5 weeks and most preferably less than 4 weeks after transformation.

Preferably, all of the selection steps are carried out at 21-32° C., more preferably at about 28° C.

In some embodiments, the process comprises
regenerating mature somatic embryos to produce shoots/roots,
preferably using a light/dark cycle.

The regeneration step is primarily used to initiate shoot formation.

Shoots are usually then transferred to root-inducing medium for root formation.

The regenerating step is preferably carried out for 2-15 weeks.

In embodiments of the invention where the plant tissue are immature embryos, the regenerating step is preferably 3-5 weeks, more preferably about 4 weeks.

In embodiments of the invention where the plant tissue are calli, the regenerating step is preferably 6-12 weeks, more preferably about 8 weeks.

In embodiments of the invention where the plant tissue are immature embryos, the regeneration step preferably starts with 2-4 days of continuous light, more preferably about 3 days continuous light.

The regenerating step is preferably carried out under a light/dark cycle.

Preferably, out of a 24 hour cycle, the light is on for 14-18 hours, more preferably on for about 16 hours.

Preferably, out of a 24 hour cycle, the dark is for 6-10 hours, more preferably for about 8 hours.

The light/dark cycle is preferably carried out for 2-8 days, more preferably for about 6 days.

The regenerating step is preferably carried out at 22-30° C., more preferably at about 25° C.

After the regeneration step, the transformed embryos are preferably maintained under a 16 hour light/8 hour dark cycle indefinitely. The temperature is preferably maintained at about 25° C.

The genetic construct may further comprise one or more promoters. It may also comprise one or more terminators.

The transgene and the selection gene may have the same or different promoters and the same or different terminators.

The promoter must be one that is operable in the selected plant cell or plastid. The promoter is one which is capable of initiating transcription of the transgene. It may also be necessary for it to be capable of initiating the transcription of the nucleotide sequence encoding an auxin biosynthetic polypeptide, in cases where the gene encoding the auxin biosynthetic polypeptide does not contain its own promoter. The promoter might, for example, be one derived from a plant or bacterial gene. Preferably, the promoter is plant specific.

Examples of suitable promoters include PpsbA, CIpP, RbcL and Prrn promoters.

Preferably, the promoter is a Prrn promoter (e.g. Plastidic ribosomal RNA (rrn) operon promoter (nt 59034-59303, accession Z00044 Nicotiana tabacum chloroplast genome DNA) or a Prrn promoter (nt 95161-96651, accession no. NC_001666.2 Zea mays chloroplast genome DNA).

In some embodiments, the promoter is an inducible promoter. This allows inducible, controlled expression of the selection gene(s). For example, the inducible promoter may be inducible by IPTG, e.g. the PrrnL promoter. Other inducible promoters include those inducible by light, dark, ethanol, drought, metals, pathogens, growth regulators, heat, cold, galactose and other sugars. Alternatively, the promoter is a high-expression level promoter.

The terminator may be a plant terminator or a bacterial terminator, inter alia.

Examples of suitable terminators include those of rrn, psbA, rbcL and T7.

The preferred terminator is a TrbcL terminator (e.g. Ribulose-1,5-Bisphosphate Carboxylase/Oxygenase polyA addition sequence (nt 102539-102685, accession Z00044 Nicotiana tabacum chloroplast genome DNA).

In yet other embodiments, the genetic construct includes a second selectable marker gene and/or a nucleotide sequence which confers resistance to an antibiotic.

The selection gene may form part of an Excision Cassette, wherein the Excision Cassette is excised from the plant genome after selection.

Such excision may involve the use of site-specific recombination elements/site-specific recombinases.

Examples of site-specific recombination elements/site-specific recombinases include Cre-lox, the FLP-FRP system from Saccharomyces cerevisae (O'Gorman S, Fox D T, Wahl G M. (1991) Recombinase-mediated gene activation and site-specific integration in mammalian cells. Science. 25, 1351-1555.), the GIN/gix system from bacteriophage Mu (Maeser S and Kahmann R. (1991) The Gin recombinase of phage Mu can catalyse site-specific recombination in plant protoplasts. Mol Gen Genet. 230, 170-176.) or the R/RS system from Zygosaccharomyces rouxii (Onouchi H, Yokoi K, Machida C, Matsuzaki H, Oshima Y, Matsuoka K, Nakamura K, Machida Y. (1991) Operation of an efficient site-specific recombination system of Zygosaccharomyces rouxii in tobacco cells. Nucleic Acids Res. 19, 6373-6378.).

The preferred recombination site is lox in combination with the recombinase Cre. Preferably, the recombinase sequence used is a cDNA sequence encoding a Cre polypeptide.

Once the Excision Cassette has been excised, an appropriate promoter should then be capable of driving the expression of the transgene, leading to the accumulation of the product of the transgene in the plant cells. The product of the transgene may be purified or isolated from the plant cell by any suitable means.

In a preferred embodiment, the invention provides a process for producing somatic plant embryos, the process comprising the steps:
(i) initiating cell differentiation from immature plant embryos
   on a callusing medium comprising 2,4-D for approx. 2 days in the dark;
(ii) pre-culturing the immature plant embryos
   on an osmotic medium for approx. 4 hours in the dark;
(iii) transforming the immature plant embryos with a genetic construct
   (preferably using a biolistic transformation method),
   wherein the genetic construct comprises a transgene
   and a gene encoding one or more auxin biosynthetic polypeptides
   (preferably iaaH/iaaM),
   and then returning the immature plant embryos to the dark for 48 hours;
(iv) culturing the bombarded immature plant embryos
   on a callusing medium comprising 2,4-D in the dark for 7 days;
(v) selecting for transformed immature plant embryos on a medium lacking 2,4-D in the dark at about 28° C. for about 7 days, followed by continuous light for about 3 days;
(vi) regenerating mature somatic embryos to produce shoots/roots,
   using a 16 hour light/8 hour dark cycle for 6 days.

Preferably, the plant is maize.

Preferably, the above steps are carried out in order without significant intervening steps, or without a gap of 12-24 hours between any of the steps.

In a further preferred embodiment, the invention provides a process for producing a transformed plant, the process comprising the steps:
(i) initiating cell differentiation from immature plant embryos to produce plant calli using a callusing medium preferably comprising 2,4-D for 6-8 weeks in the dark;
(ii) pre-culturing the plant calli on an osmotic medium for 4 hours in the dark;
(iii) transforming the plant calli with a genetic construct
   using a biolistic transformation method,
   wherein the genetic construct comprises a transgene
   and a gene encoding one or more auxin biosynthetic polypeptides
   (preferably iaaH/iaaM);
(iv) culturing the bombarded plant calli
   on a osmotic medium in the dark for approx. 24 hours;
(v) selecting for transformed plant calli on media on a medium lacking 2,4-D in the dark at about 28° C. for about 7 days;
(vi) regenerating plant calli to produce one or more transformed plants
   using a 16 hour light/8 hour dark cycle at about 28° C.

Preferably, the plant is maize.

Preferably, the above steps are carried out in order without significant intervening steps, or without a gap of 12-24 hours between any of the steps.

The invention also provides a process for making a transgene product, comprising the process for producing a transformed plant embryos, as described hereinbefore, and additionally comprising purifying the transgene product from the regenerated plants.

The invention also provides a transgene product obtained or obtainable by a process of the invention.

Additionally, the invention provides a transformed plant embryo or transformed plant obtainable or obtained using a process of the invention.

### BRIEF DESCRIPTION OF THE FIGURES

FIG. 1 shows schematic diagrams of the targeting region in the maize plastid genome and the resulting transplastome following integration of the pAD001 transgene cassette. The transgenes are targeted to the region between the 5′rps12 and clpP genes. Construction of the plastid transformation vector pAD001: 5′rps12 homologous recombination sequence (nt 68231-69454, accession NC_001666.2 Zea mays chloroplast genome DNA); PrrnT7g10L: Plastidic ribosomal RNA (rrn) operon promoter (nt 139983-14065, accession Z00044 Nicotiana tabacum chloroplast genome DNA) fused to the leader sequence of bacteriophage T7 gene 10; iaaM gene from Agrobacterium tumefaciens; IEE, putative processing element (Zhou, F., Karcher. D. and Bock., R. Identification of a plastid intercistronic expression element (IEE) facilitating the expression of stable translatable monocistronic mRNAs from operons. The Plant Journal (2007) 52, 961-972); iaaH gene from Agrobacterium tumefaciens; TpsbA: psbA polyA addition sequence (nt 141-536, accession Z00044 Nicotiana tabacum chloroplast genome DNA); clpP; homologous recombination sequence (nt 69455-71184, accession NC_001666.2 Zea mays chloroplast genome DNA).

FIG. 2 shows images of regeneration in maize following bombardment of immature maize embryos. (A) Immature maize embryos 1 day post bombardment with pAD001 construct. (B) Immature maize embryos 7 days post bombardment on selection medium (−) 2,4-D in the dark. A number of immature maize embryos remain white and continue to proliferate and grow as callus. Browning of other immature maize embryos signifies death. (C) Green calli (red circles) visible 2½ to 3 weeks post bombardment on selection medium following the introduction of light. (D) Immature maize embryos on the control plate 2 weeks post bombardment on selection medium.

FIG. 3 shows images of regeneration in maize following bombardment of immature maize embryos. (A) Following the introduction of light, green calli is visible 2½ weeks post bombardment of immature maize embryos with pAD001 construct. (B) Immature maize embryos bombarded with an empty vector (control) turn brown and die 2 weeks post bombardment.

FIG. 4 shows images of regeneration in maize following bombardment of callus derived from immature maize embryos. (A) Calli derived from immature maize embryos 1 day post bombardment with pAD001 construct on selection media (−) 2,4-D. (B) Green calli is visible 4½ weeks post bombardment following 4 weeks incubation in the dark and 3 days under a 16/8 hr light dark cycle. (C) Green calli approximately 4½ weeks following bombardment with pAD001 construct.

FIG. 5 shows PCR analysis confirming the presence of the iaaM-iaaH transgenes. (A) Schematic diagram showing the approximate annealing position of the iaaM-F and iaaH-R primers used to confirm the presence of the iaaM-iaaH transgenes. Amplification of a PCR product confirmed the presence of the iaaM-iaaH transgenes in putative transformed calli derived from immature maize embryos (B) and in putative transformed immature maize embryos (C).

FIG. 6 shows PCR analysis confirming the correct integration of the pAD001 transformation vector into the left homologous recombination border region of the maize plastome. (A) Schematic diagram showing the approximate annealing position of the Ext-F and iaaM-R primers used to confirm the correct integration of the pAD001 transformation vector into the left homologous recombination border region of the maize plastome. (B) Amplification of a PCR product confirmed the correct integration of the pAD001 transformation vector in putative transformed calli derived from immature maize embryos.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

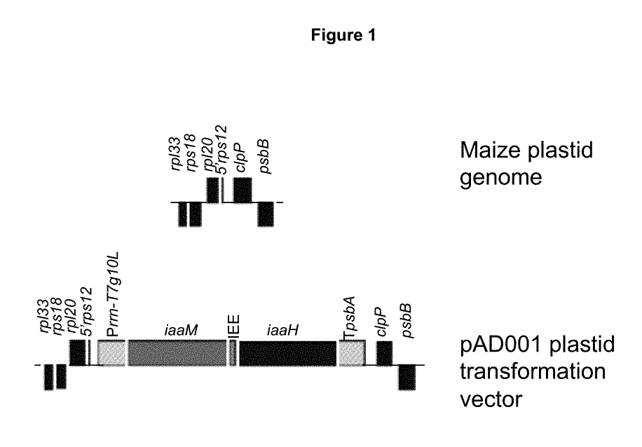

The plastid transformation vector pAD001 was constructed as detailed in FIG. 1 using a 1223 bp homologous recombination sequence (nt 68231-69454) and a 1729 bp homologous recombination sequence (nt 69455-71184) from the chloroplast genome from Zea mays (Maier, R. M., Neckermann, K., Igloi, G. L. and Kossel, H. (1995). Complete sequence of the maize chloroplast genome: gene content, hotspots of divergence and fine tuning of genetic information by transcript editing. J. Mol. Biol. 251 (5), 614-628 (1995)) on either side of the gene cassette.

For proof-of-principle purposes, the iaaM-iaaH transgene cassette was cloned in between the homologous recombination sequences to generate pAD001 (FIG. 1). This vector was then bombarded into both immature maize embryos and maize callus as detailed in Appendix 1. For the transformation of immature maize embryos, ears were collected 10-13 days after pollination from greenhouse grown Hi II plants (A188×B73 origin, Armstrong, C. L., Green, C. E., and Phillips, R. L. (1991) Development and availability of germplasm with high Type II culture formation response. Maize Genetics Coop Newsletter 65: 92-93.). Ears were sterilized in 30-50% commercial bleach containing 3 drops of Tween 20 for 30 minutes and washed in sterilized water three times. Immature zygotic embryos were excised from the ears and placed embryo axis side down on N6E callus initiation medium (containing 2,4D) for 2 days in the dark as scutellum-derived callus is most likely the producer of transgenic events. For the transformation of Hi II Type II callus, ears were sterilized as described previously. Immature maize embryos were excised and placed on N6E callus initiation medium (containing 2,4D) for six to eight weeks in the dark.

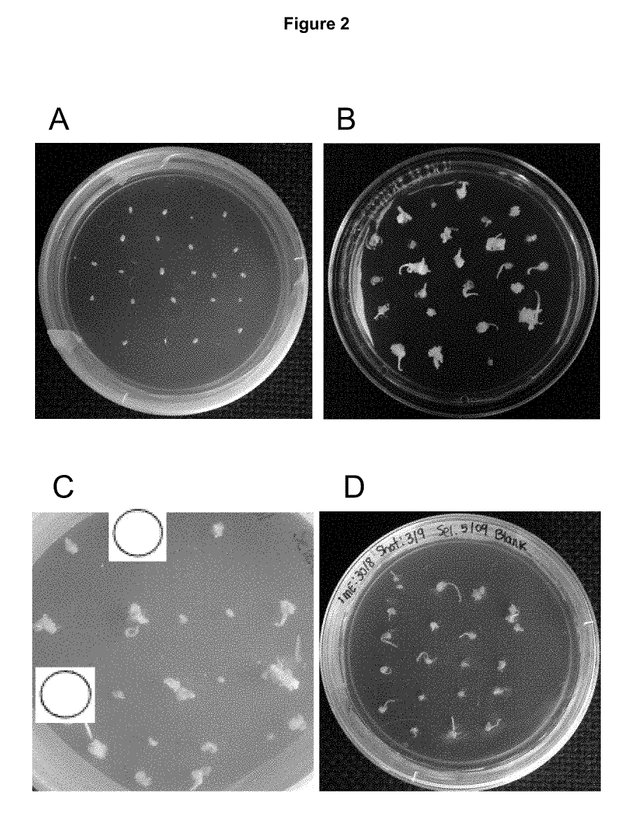

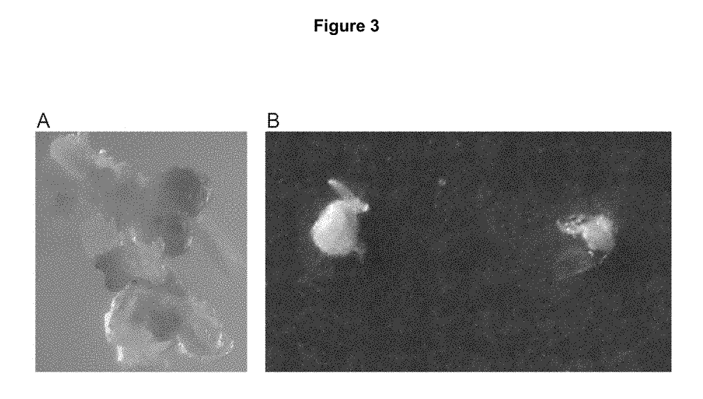

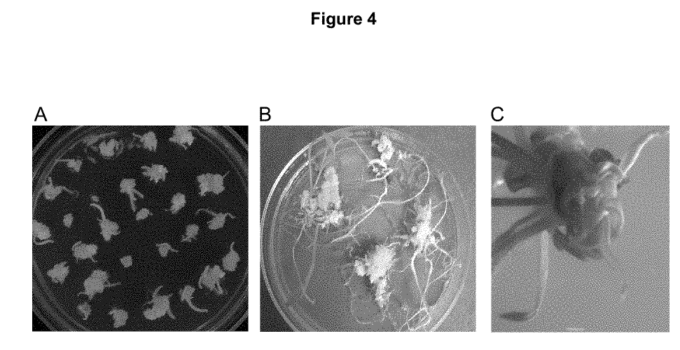

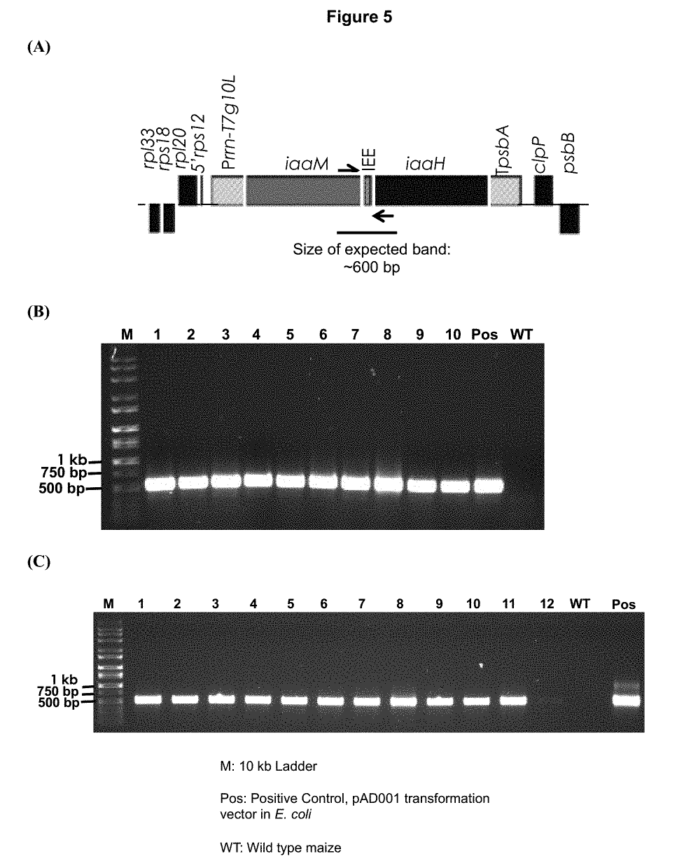

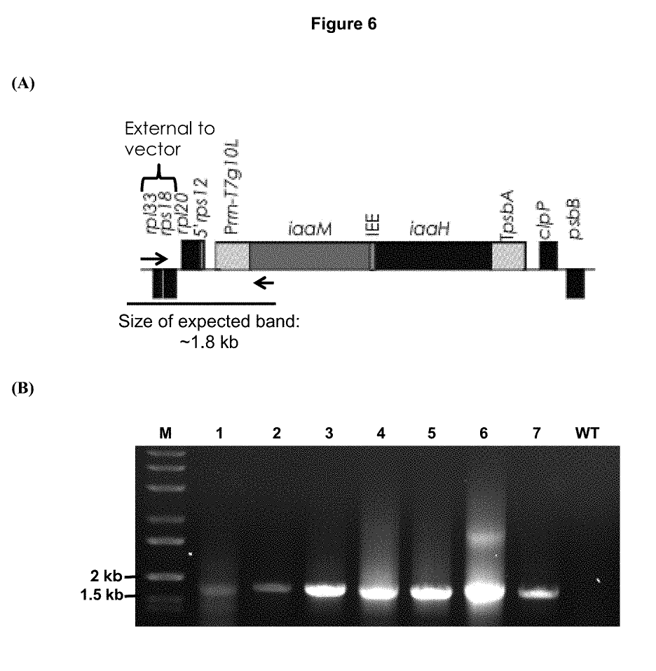

The pAD001 construct was transformed into plastids using the protocol shown in Appendix 1 followed by auxin mediated selection and regeneration (FIGS. 2A, 2B, 3A, 3B, 4A and 4B). PCR verification of iaaM-iaaH presence in maize cells was carried out on genomic DNA prepared from regenerated maize calli (FIG. 2C, 4C) and the primers iaaM-F and iaaH-R, which span the iaaM-iaaH junction in the transgene cassette (FIG. 5A). FIG. 5B and FIG. 5C show the presence of the iaaM-iaaH transgene in putative transgenic callus derived from immature maize embryos and immature maize embryos respectively. To determine whether plastid integration had occurred, PCR analysis was carried out using the primers Ext-F (which anneals to sequence external to the homologous recombination sequence on the transformation sequence) and iaaM-R, which anneals internal to the iaaM transgene (FIG. 6A). FIG. 6B shows the correct integration of the transformation vector into the plastid genome on the left homologous recombination side in callus derived from immature maize embryos.

### Example 2: Expression of Proteins Conferring Abiotic Stress Resistance

Abiotic stresses such as drought, salinity and temperature can be very detrimental to plants because of their sessile existence and can result in severe reduction in crop yields worldwide. The described system allows for the introduction and selection of transgenes, which can confer tolerance to abiotic stresses, in the maize plastid genome.

Transgenes e.g. the betaine aldehyde dehydrogenase gene, which confers tolerance to salinity and trehalose phosphate synthase, which confers drought tolerance, can be inserted into the pAD001 vector (FIG. 1) between the PrrnT7g10L and the TpsbA sequence. The construct is transformed into plastids using the protocol shown in Appendix 1 followed by auxin mediated selection and regeneration. The use of this system allows for a high level of transgene containment as plastids are predominantly maternally inherited in most crops. Maternal inheritance stops the escape of plastid genes and transgenes by pollen transmission, which is a significant advantage over nuclear transformation. In addition, environmental as well as health concerns in relation to the integration of antibiotic resistance genes in transformed plants is eliminated as this selection system does not contain an antibiotic selectable marker resulting in improved safety.

### Example 3: Expression of Proteins Conferring Biotic Stress Resistance

Biotic stresses such as bacterial, viral and fungal pathogens in addition to weeds and pests affect crop yields yearly and can result in significant financial losses to both farmers and industry alike.

As described in Example 2, biotic stress resistant transgenes e.g. B. thuringiensis (Bt) cry1A(c), may be inserted into the pAD001 vector followed by transformation, selection and regeneration as described previously.

### Example 4: Expression of Proteins Conferring Both Abiotic and Biotic Stress Resistance

A major limitation that both farmers and scientists face in crop production worldwide is the loss of up to 30-60% crop yield each year due to a combination of both biotic and abiotic stresses (Dhlamini. Z., Spillane. C., Moss. J P., Ruane. J., Urquia. N., Sonnino. A., (2005). Status of research and applications of crop biotechnologies in developing countries: Preliminary assessment, Roma, Food and Agriculture Organization of the United Nations [ISBN 92-5-105290-5]). As described in Examples 2 and 3, a combination of transgenes conferring both abiotic and biotic stress resistance may be inserted into the pAD001 vector, followed by transformation, selection and regeneration as previously described.

### Example 5: Removal of the Auxin Biosynthetic Genes Post Selection

An increase in auxin due to the integration of the iaaM-iaaH transgene cassette may alter the growth characteristics of transformed plant species. To avoid this issue, the system described in Example 1 can be combined with a system for eviction such as a RIRS system from Zygosaccharomyces rouxii, Flp/frt from Saccharomyces cerevisiae, and Gin/gix from bacteriophage Mu removing the iaaM-iaaH transgene cassette and thus eliminating the problem.

### Example 6: Generation of Whole Transformed Plants

The generation of whole transformed plants may be achieved by the following protocol. Transformation vectors containing the iaaM-iaaH gene cassette as a selectable marker are constructed and then bombarding into maize tissue as described above. Following the selection and confirmation of putative transformed calli, as described above, shoot regeneration could then be achieved using a cocktail of plant growth regulators (e.g. cytokinins etc.) to promote organogenesis. Alternatively, an antibiotic resistant gene may be incorporated in addition to the auxin genes (iaaM-iaaH) and the use of a two step selection system, first utilizing the iaaM-iaaH gene cassette for initial selection of transformants in the dark and secondly utilizing the antibiotic resistance gene once the calli are moved into the light.

### APPENDIX 1

### Protocol for Chloroplast Transformation

### Time Course

The standard procedures produce transformed plants in 3-5 months.

### Equipment Set Up

### Helium Gun Bio-Rad PDS 1000

Rupture disk PSI: 900
Gap between rupture disk retaining cap and macrocarrier over cover lid: ¼″
Spacer rings below stopping screen support: 2
Level of macrocarrier launch assembly: 1 (from top)
Level of Petri dish holder: 3 (from top)
Vacuum inflow rate: Maximum
Vacuum release rate: attenuate the release so it approximates the speed of vacuum inflow.

### Stock Solutions

2.5 M CaCl₂ filter sterilized
0.1 M Spermidine Free Base in sterilized H₂O
dH₂O
DNA at 1 μg/μl in dH₂O or 1× TE
100% Ethanol
70% Isopropanol

### Consumables

900 PSI rupture disks
Stopping screens
Macrocarriers
Gold particles

### Preparation of the DNA-Gold Particle Mix

50 mg of gold particles are suspended in 1 ml of 100% ethanol as stock
Take 0.25 ml of Gold stock suspension and centrifuge for 5 seconds. Remove ethanol and wash three times with sterile distilled H₂O, centrifuging 3 minutes between washings.
Resuspend Gold in 0.25 ml dH₂O.
Aliquot 50 μl of Gold-H₂O suspension into Eppendorf tubes.
Into each Eppendorf tube add the following in succession:
10 μl DNA at 1 μg/μl
50 μl of 2.5 M CaCl₂
20 μl of 0.1 M Spermidine free base
Vortex for 5 minutes at highest speed.
Add 200 μl of 100% ethanol to each tube.
Centrifuge at 3000 rpm for 10 seconds.
Remove as much supernatant as possible and rinse pellet in 100% ethanol once, centrifuging at 3000 rpm for 10 seconds.
Resuspend pellet in 30 μl 100% ethanol (makes 4-5 shots). Store mixture on ice.

### Preparing the Biolistic Gun and Consumables

Sterilize the gun vacuum chamber and surfaces with 70% ethanol.
Sterilize the stopping screens and macrocarrier holders by autoclaving.
Sterilize the rupture disks in 70% isopropanol.
Sterilize the macrocarriers in 100% ethanol. Air dry in hood.
Open helium tank. Set the helium tank regulator to 1100 psi (or 200 psi above the rating of the rupture disk.

### Bombardment

Particle bombardment was carried out using a biolistic PDS-1000/He gun (Bio-Rad).
Place sterile macrocarriers into the macrocarrier holders.
Pipet 5 μl of vortexed gold/DNA mixture onto the center of each sterile macrocarrier and leave at room temperature for 10 minutes.
Insert a sterile rupture disc into the recess of the retaining cap and tightly screw onto the gas acceleration tube.
Place a sterile stopping screen on the support and install the macrocarrier holder on the rim of the fixed nest.
Screw the macrocarrier lid onto the assembly and place the macrocarrier launch assembly in the top slot inside the bombardment chamber.
Place the target shelf at the desired distance, 6 cm from the macro-projectile stopping screen (three from top) and place the Petri dish containing the target tissue on it.
Open the helium tank to 1100 psi (200 psi greater than the capacity of the rupture disc).
Close the door of the gene gun; evacuate the chamber to 28 Hg (inches of mercury) and hold at this vacuum.
Press the fire button and release once the rupture disc has burst.
Vent the chamber, remove the Petri dish and repeat the procedure for subsequent shots.
At the end of the experiment, turn off the helium tank. Pull a vacuum in the gun to release the remaining helium through the gun and then turn off the helium regulator.
   The key to successful bombardment is usually in the spread of particles on the macrocarrier. The gold-DNA mixture should be spread evenly over the center of the macrocarrier. The resulting spread should be void of any clumps, which can result in an increased frequency of cell death. Each 30 μl gold-DNA mix usually gives 4-5 bombardments.

### Maize Preparation and Regeneration Media

### N6E (Callus Initiation):

4 g/L N6 salts (Chu et al., 1975)
1 ml/L (1000×) N6 vitamin stock
2 mg/L 2,4-D
100 mg/L myo-inositol
2.76 g/L proline
30 g/L sucrose
100 mg/L casein hydrolysate
2.5 g/L agar,
20 pH 5.8 and autoclave
Silver nitrate (25 μM) added after autoclaving.

### N6OSM (Osmotic Medium):

4 g/L N6 salts
1 ml/L N6 vitamin stock
2 mg/L 2,4-D
100 mg/L myo-inositol
0.69 g/L proline
30 g/L sucrose
100 mg/L casein hydrolysate
36.4 g/L sorbitol
36.4 g/L mannitol (Vain et al, 1993)
2.5 g/L agar
pH 5.8 and autoclave.
Silver nitrate (25 μM) added after autoclaving.

### N6S (Selection Minus Auxin (2,4-D)):

4 g/L N6 salts
1 ml/L N6 vitamin stock
100 mg/L myo-inositol
30 g/L sucrose
2.5 g/L agar
pH 5.8 and autoclave.
Silver nitrate (25 μM) added after autoclaving.

### Tissue Culture

### Pre-Bombardment

Dehusk ear. Cut off and discard top 1 cm of ear. Place ear into a clean beaker.
Add ˜700 ml of 70% ethanol (or enough to cover ear), swirl for 1-2 minutes and discard the ethanol. Wash the ear three times with sterile distilled H₂O.
Add ˜700 ml sterilizing solution (30-50% commercial bleach in water and 3 drops of Tween 20) to cover ear. Throughout the 30 minute disinfection, swirl the ears to dislodge air bubbles for thorough surface sterilization of ear.
Pour off the bleach solution, rinse the ears three times in sterile distilled H₂O and the ears are ready for embryo dissection.
In a large (150×15 mm) sterile petri-plate, cut off the kernel crowns (the top 1-2 mm) with a sharp scalpel blade. Re-sterilize the forceps and scalpels intermittently throughout this protocol to avoid contamination.
Excise the embryos by inserting the narrow end of a narrow pointed forceps between the endosperm and pericarp at the basipetal side of the kernel. The embryo is gently coaxed onto the tip of the forceps and plated with the embryo-axis side down (scutellum side up) onto the N6E media (approximately 30 embryos/plate).
Wrap the plate with vent tape and incubate for 2 or 3 days (if transforming immature maize embryos) or 6-8 weeks (if transforming callus derived from immature maize embryos) at 28° C. in the dark.
Four hours prior to bombardment, use sterile forceps to transfer the embryos or calli onto the osmotic medium (N6OSM), (Vain et al., 1993). Center the embryos or calli in the center of the plate. Embryos should be facing scutellum side up at bombardment since it is from this surface that subsequent callus initiation begins and from which transformed cells are then selected.

### Post Bombardment

The bombarded embryos or calli (still on N6OSM) are gently wrapped with vent tape and incubated in 28° C. in the dark.

### Selection for Stable Transformed Events (Immature Maize Embryos)

The next day (16-20 hours after bombardment), embryos are transferred off the N6OSM and onto N6E media to continue callus initiation. Embryos are again oriented scutellum side up and plates are wrapped with vent tape.
After 7 days, embryos are transferred to selection medium (lacking 2,4D) and placed in the dark at 28 degrees for a further 7 days.
Embryo selection plates are then placed under continuous light for 3 days, followed by 16/8 hour light/dark cycle for a further 6 days.
Green calli are visible 3 days after being placed in light (2 weeks post bombardment).
   Selection for Stable Transformed Events (Callus Derived from Immature Maize Embryos)
24 hours post bombardment; Calli are transferred to selection medium (without 2,4-D) and placed in the dark at 28° C. for 7 days.
Four weeks post bombardment; plates removed from the dark and placed under a 16/8 hour light/dark cycle at 28° C. Green calli begin to appear 3 days later (approximately 4.5 weeks post bombardment).

### Regeneration Protocol

A standard protocol is used as for nuclear transformation.

1. A process for producing a transformed plant tissue, the process comprising the steps:
(i) transforming plant tissue with a genetic construct,
wherein the genetic construct comprises a transgene and a selection gene,
wherein the selection gene encodes an auxin biosynthetic polypeptide; and
(ii) selecting for transformed plant tissue using a light/dark cycle on media which is lacking plant auxin.

2. A process as claimed in claim 1, wherein the process comprises:
initiating cell differentiation from a plant tissue; and/or
pre-culturing the plant tissue on osmotic medium, prior to the transforming step.

3. A process as claimed in claim 1, wherein the process comprises:
a post-transformation recovery interval prior to the selection step.

4. A process as claimed in claim 1, wherein the process comprises:
regenerating mature somatic embryos to produce shoots/roots,
preferably on media which is lacking plant auxin using a light/dark cycle.

5. A process as claimed in claim 1, wherein the transforming step is carried out using a biolistic transformation method.

6. A process for producing somatic plant embryos, the process comprising the steps:
(i) initiating cell differentiation from immature plant embryos on a callusing medium comprising auxin;
(ii) pre-culturing the immature plant embryos on an osmotic medium in the dark;
(iii) transforming the immature plant embryos with a genetic construct using a biolistic transformation method, wherein the genetic construct comprises a transgene and a gene encoding one or more auxin biosynthetic polypeptides;
(iv) optionally culturing the immature plant embryos on a callusing medium;
(v) selecting for transformed immature plant embryos on media which is lacking plant auxin on a medium lacking 2,4-D in the dark; and
(vi) selecting mature somatic embryos on media which is lacking plant auxin using an optional continuous light cycle and then using a light/dark cycle, wherein
the optional continuous light cycle is for about 2-4 days, and
the light/dark cycle is approx. 16 hour light/8 hour dark cycle for 2-8 days.

7. A process for producing a transformed plant, the process comprising the steps:
(i) initiating cell differentiation from immature plant embryos to produce plant calli on a callusing medium comprising auxin;
(ii) pre-culturing the plant calli on an osmotic medium in the dark;
(iii) transforming the plant calli with a genetic construct using a biolistic transformation method, wherein the genetic construct comprises a transgene and a gene encoding one or more auxin biosynthetic polypeptides;
(iv) optionally culturing the bombarded plant calli on an osmotic medium;
(v) selecting for transformed plant calli on media which is lacking plant auxin on a medium lacking 2,4-D in the dark;
(vi) selecting plant calli on media which is lacking plant auxin using a light/dark cycle, wherein the light/dark cycle is approx. 16 hour light/8 hour dark cycle for 2-8 days; and
(vii) optionally regenerating a transformed plant from the calli.

8. A process as claimed in claim 1, wherein the plant is a monocot or a dicot.

9. A process as claimed in claim 1, wherein the plant is selected from the group consisting of cereals, legumes, oil crops, cash crops, vegetable crops, fruit trees, nut trees, beverages, timber trees, mosses and duckweed.

10. A process as claimed in claim 1, wherein the plant is maize.

11. A process as claimed in claim 9, wherein the plant tissue is a plant embryo or plant callus.

12. A process as claimed in claim 1, wherein the genetic construct is targeted to plastids within the plant tissue.

13. A process as claimed in claim 1, wherein the transgene encodes one or more antibodies, antibiotics, herbicides, vaccine antigens, enzymes, enzyme inhibitors or design peptides.

14. A process as claimed in claim 1, wherein the auxin biosynthetic polypeptide is selected from the group consisting of iaaH/iaaM, AMI1, TAA1, TAR1, TIR2, YUC, AAO1, CYP79B2 and TDC.

15. A process as claimed in claim 1, wherein the process comprises:
regenerating mature somatic embryos or plants on selection media using a light/dark cycle.

16. A process for making a transgene product, the process comprising a process for producing a transformed plant tissue as claimed in claim 1, and additionally comprising purifying the transgene product from the regenerated plant.

17. A transgene product obtained or obtainable by a process as claimed in claim 1.

18. A process as claimed in claim 2, wherein the process comprises:
a post-transformation recovery interval prior to the selection step.

19. A process as claimed in claim 2, wherein the process comprises:
regenerating mature somatic embryos to produce shoots/roots,
preferably on media which is lacking plant auxin using a light/dark cycle.

20. A process as claimed in claim 3, wherein the process comprises:
regenerating mature somatic embryos to produce shoots/roots,
preferably on media which is lacking plant auxin using a light/dark cycle.

21. A process as claimed in claim 18, wherein the process comprises:
regenerating mature somatic embryos to produce shoots/roots,
preferably on media which is lacking plant auxin using a light/dark cycle.

22. A process as claimed in claim 2, wherein the transforming step is carried out using a biolistic transformation method.

23. A process as claimed in claim 3, wherein the transforming step is carried out using a biolistic transformation method.

24. A process as claimed in claim 18, wherein the transforming step is carried out using a biolistic transformation method.

25. A process as claimed in claim 4, wherein the transforming step is carried out using a biolistic transformation method.

26. A process as claimed in claim 19, wherein the transforming step is carried out using a biolistic transformation method.

27. A process as claimed in claim 21, wherein the transforming step is carried out using a biolistic transformation method.

28. A process as claimed in claim 6, wherein the plant is a monocot or a dicot.

29. A process as claimed in claim 7, wherein the plant is a monocot or a dicot.

30. A process as claimed in claim 6, wherein the plant is selected from the group consisting of cereals, legumes, oil crops, cash crops, vegetable crops, fruit trees, nut trees, beverages, timber trees, mosses and duckweed.

31. A process as claimed in claim 7, wherein the plant is selected from the group consisting of cereals, legumes, oil crops, cash crops, vegetable crops, fruit trees, nut trees, beverages, timber trees, mosses and duckweed.

32. A process as claimed in claim 30, wherein the plant tissue is a plant embryo or plant callus.

33. A process as claimed in claim 31, wherein the plant tissue is a plant embryo or plant callus.

34. A process as claimed in claim 10, wherein the plant tissue is a plant embryo or plant callus.

35. A process as claimed in claim 6, wherein the genetic construct is targeted to plastids within the plant tissue.

36. A process as claimed in claim 7, wherein the genetic construct is targeted to plastids within the plant tissue.

37. A process as claimed in claim 6, wherein the transgene encodes one or more antibodies, antibiotics, herbicides, vaccine antigens, enzymes, enzyme inhibitors or design peptides.

38. A process as claimed in claim 7, wherein the transgene encodes one or more antibodies, antibiotics, herbicides, vaccine antigens, enzymes, enzyme inhibitors or design peptides.

39. A process as claimed in claim 6, wherein the auxin biosynthetic polypeptide is selected from the group consisting of iaaH/iaaM, AMI1, TAA1, TAR1, TIR2, YUC, AAO1, CYP79B2 and TDC.

40. A process as claimed in claim 7, wherein the auxin biosynthetic polypeptide is selected from the group consisting of iaaH/iaaM, AMI1, TAA1, TAR1, TIR2, YUC, AAO1, CYP79B2 and TDC.

41. A process as claimed in claim 6, wherein the process comprises:
regenerating mature somatic embryos or plants on selection media using a light/dark cycle.

42. A process as claimed in claim 7, wherein the process comprises:
regenerating mature somatic embryos or plants on selection media using a light/dark cycle.

43. A process for making a transgene product, the process comprising a process for producing a transformed plant tissue as claimed in claim 6, and additionally comprising purifying the transgene product from the regenerated plant.

44. A process for making a transgene product, the process comprising a process for producing a transformed plant tissue as claimed in claim 7, and additionally comprising purifying the transgene product from the regenerated plant.
